(19) United States
(12) Plant Patent Application Publication
(10) Pub. No.: US 2003/0150029 P1
(43) Pub. Date: Aug. 7, 2003

(54) Symphoricarpos x doorenbosii named 'ARISO'

(76) Inventor: C.M. Arisz, Beverwijk (NL)

Correspondence Address: CHRISTIE, PARKER & HALE, LLP, 350 WEST COLORADO BOULEVARD, SUITE 500, PASADENA, CA, 91105, US

(21) Appl. No.: 10/052,646

(22) Filed: Jan. 18, 2002

(51) Int. Cl. A01H 5/00

(52) U.S. Cl. PLT/226

(57) ABSTRACT

A new and distinct ‘Symphoricarpos’ x doorenbosii plant with attractive red-purple fruit.

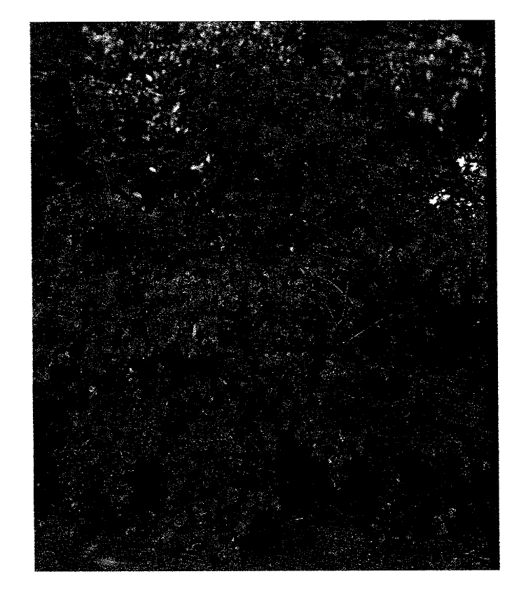

### BACKGROUND OF THE INVENTION

The present invention relates to a new and distinct variety of ‘Symphoricarpos’ x doorenbosii named ‘ARISO’. The new variety was created by crossing ‘Symphoricarpos’ x doorenbosii The varietal denomination of the new variety is ‘ARISO’.

### SUMMARY OF THE INVENTION

Among the novel characteristics possessed by the new variety which distinguishes it from its parents and all other varieties of which I am aware, are its attractive red-purple fruit.

Asexual reproduction by propagation of vegetative cuttings of the new variety as performed in The Netherlands shows that the foregoing and other distinguishing characteristics come true to form and are established and transmitted through succeeding propagations.

### COMPARISON WITH PARENTS

Fruit of ‘ARISO’ differ from the rest of ‘Symphoricarpos’ x doorenbosii ‘Mother of Pearl’ in having bright red-purple color with lighter blotches whereas ‘Mother of Pearl’ have lighter to almost white fruit.

### BRIEF DESCRIPTION OF ILLUSTRATION

The accompanying photograph illustrates a typical specimen of the fruit and foliage of the new variety depicted in color as nearly true as it is reasonably possible to make in a color illustration of this character.

### DESCRIPTION OF THE NEW VARIETY

The following is a detailed description of the new variety with color terminology, references and/or values in accordance with The Royal Horticultural Society Colour Chart (RHSCC). The terminology used in the color descriptions herein refers to plate numbers in the aforementioned color chart except where common terms of color definition are employed. Phenotypic expression may vary with environmental, cultural and climatic conditions, as well as differences in conditions of light and soil.

The following observations are made of specimens grown outdoors in The Netherlands. The range of average Summer day temperature is ±18° C. and average Summer night temperature is ±10° C. No photoperiodic treatments or growth retardants were used.

| | |
|---|---|
| PLANT | |
| Crop Time: | Approximately 1 year from |
| | rooted cuttings; field grown |
| Growth Habit: | Broad upright/spreading |
| | with arching branches |
| Vigor: | Growth rate/vigor is |
| | moderate to vigorous |
| Plant Shape: | Broad, flattened globe-shaped |
| Size: | Height about 1 m; diameter or |
| | area of spread about 1.25 m |
| Branching Habit: | Free branching: main shoots |
| | appear from base |
| Pinching Required: | No pinching required |
| Lateral Branches: | Average quantity 16; length |
| | average 16 cm (shorter |
| | towards the top); diameter |
| | average 1.5 mm |
| Internode: | Average length 4.5 cm |
| Stem: | Stems slightly exfoliating |
| | when older (only at the |
| | base); color brown, near 175A |
| | to 175B |
| Pubescence: | No |
| Leaves | |
| Arrangement: | Opposite; singly borne |
| Quantity per lateral branch: | Average 12 (between 8 to 14) |
| Shape: | Broad—elliptic to almost |
| | round; tip sometimes very |
| | short; base obtuse to |
| | slightly alternate |
| Size: | Length on main stems, average |
| | 3.5 cm, on lateral branches, |
| | average 2 cm; width on main |
| | stems average 3.5 cm, on |
| | lateral branches average 2.4 cm |
| Texture: | Slightly rugose, dull |
| Pubescence (which side): | No leaf pubescence |
| Margin: | Entire (rarely with 1 or 2 |
| | deep crenate lobes |
| Venation Pattern: | Pinnate |
| Color: | Immature leaflets upper side |
| | dark-green, near 139A; under |
| | side greyish-green, near |
| | 131A; mature leaflets upper |
| | side dark-green, near 139A, |
| | under side greyish-green, |
| | near 131A |
| Vein color: | Upper side main view green, |
| | near 143A; under side main |
| | view green near 143A |
| Petiole: | Length up to about 4 mm |
| | (average 3 mm); diameter |
| | about 1 mm; color green, near |
| | 143A |
| Durability of Foliage | High |
| to Stresses: | |
| Stipules, Tendrils, Thorns, | None |
| Spines or Prickles: | |
| FLOWER | |
| Arrangement: | Panicle |
| Form: | Campanulate |
| Habit: | Once a year |
| Quantity: | Average 20 buds and flowers |
| | per lateral stem; more than |
| | 1,000 buds and flowers per |
| | plant |
| Flowering season: | Late summer |
| Time to flower or response | One year |
| time: | |
| Rate of opening: | Approximately 20% per panicle |
| | at once |
| Fragrance: | None |
| Aspect: | Campanulate |
| Shape: | Campanulate |
| Size: | Diameter about 4 mm; depth or |
| | height about 5 mm |
| Longevity: | A few days on the plant; as |
| | cut flower, not applicable |
| Persistence: | Self cleaning |
| Petalage | |
| Appearance: | Dull, smooth |
| Texture: | Smooth |
| Arrangement: | Campanulate |
| Number | About 5 |
| Fused: | Partly fused, top ⅓rd free |
| Shape: | Oval to elliptic |
| Margin: | Entire |
| Tip: | Shallowly incised/praemorse |
| | to almost entire |
| Size: | Length about 5 mm; width |
| | about 3 mm |
| Color: | When opening upper side |
| | greenish-white near 137C, |
| | under side greyish-purple |
| | near 185D or lighter to |
| | almost white; when fully |
| | opened upper side greenish- |
| | white near 157, under side |
| | greyish-purple near 188D or |
| | lighter to almost white, |
| | color does not fade |
| Petaloids: | None |
| Sepals: | Appearance dull, smooth |
| | texture; funnel form |
| | arrangement |
| Quantity: | About 5 |
| Margin: | Entire |
| Tip: | Acuminate |
| Color: | Upper side near 143A, |
| | sometimes with small greyish- |
| | purple blotches near 184A; |
| | under side green near 143A |
| Calyx: | Funnel form shaped, length |
| | about 4 mm (including ovary); |
| | diameter about 2 mm |
| Peduncle: | Individual flowers without |
| | peduncle; angle: calyx |
| | (ovary) rachis is 45° to 90° |
| | (stem); moderate strength |
| | (flower—rachis (stem) |
| | attachment) |
| Bud: | Oval to imbricated shaped; |
| | length about 5 mm; diameter |
| | about 3 mm; color greyish- |
| | purple, near 185D or lighter |
| | to pale yellowish-green 195C |
| Rate of opening: | Approximately 20% at once per |
| | panicle |
| Stamens: | About 5 |
| Anthers: | Narrow, oblong; about 1 mm, |
| | color creamy-white (too small |
| | to be measured by RHSCC) |
| Pistils: | About 1, length about 3 mm |
| Stigma: | Club shaped, color light- |
| | green near 155C |
| Styles: | Length about 2.5 mm; color |
| | very light-green near 145D |
| Ovary: | Green near 143A, sometimes |
| | with a small greyish-purple |
| | bottom near 184A |
| Seeds: | Yes; length average about 2 |
| | mm (between 10 to 13 mm); |
| | diameter average 13 mm |
| | (between 10 to 14 mm); color |
| | red-purple near 67A to 67B |
| | often with lighter near 70B |
| | blotch |
| Disease resistance: | Average for species |

I claim:

1. A new and distinct plant of the variety substantially as shown and described.
